SEQUENCE LISTING (8 sequences)

SEQ ID NO: 1 - protein, 119 aa, synthetic construct
  REGION 1..119: note = VH sequence
DVQLQESGPGLVKPSQSLSLTCTVSGYSITSDYAWNWIRQFPGNKLEWMGYISYTLTTGYNPSLKSRISITRDSSKNQFFLQLNSVTTEDTATYYCARSGWLLPYWYFDVWGAGTTVTV

SEQ ID NO: 2 - protein, 108 aa, synthetic construct
  REGION 1..108: note = VL sequence
DVQITQSPSYLAASPGETITINCRASKSISKYLAWYQEKPGKTNELLIYSGSTLQSGIPSRFRGSGSGTDFTLTISSLEPEDFAMYYCQQHNEYTLTFGGGTKLELKR

SEQ ID NO: 3 - protein, 250 aa, synthetic construct
  REGION 1..250: note = single-chain variable fragment antibody
MDVQLQESGPGLVKPSQSLSLTCTVSGYSITSDYAWNWIRQFPGNKLEWMGYISYTLTTGYNPSLKSRISITRDSSKNQFFLQLNSVTTEDTATYYCARSGWLLPYWYFDVWGAGTTVTVSSGGGGSGGGSGGGGSDVQITQSPSYLAASPGETITINCRASKSISKYLAWYQEKPGKTNELLIYSGSTLQSGIPSRFRGSGSGTDFTLTISSLEPEDFAMYYCQQHNEYTLTFGGGTKLELKRHHHHHH

SEQ ID NO: 4 - protein, 42 aa, synthetic construct
  REGION 1..42: note = HRC4
  SITE 1: note = MISC_FEATURE - Proline is acetylated
  REGION 1..42: note = MISC_FEATURE - Two of this peptide is conjugated to one cholesterol molecule
  SITE 42: note = MISC_FEATURE - Cysteine is modified with PEG4-bisMaleimide
PPISLERLDVGTNLGNAIAKLEDAKELLESSDQILRGSGSGC

SEQ ID NO: 5 - DNA, 363 bp, synthetic construct
  misc_feature 1..363: note = VH sequence
  source 1..363: mol_type = other DNA
gatgtgcagcttcaggagtcgggacctggcctggtgaaaccttctcagtctctgtccctcacctgcactgtctctggctactcaatcaccagtgattatgcctggaactggatccggcagtttccaggaaacaaactggagtggatgggctacataagctacactcttaccactggctacaacccatctctcaaaagtcgaatctctatcactcgagactcatccaagaaccagttcttcctgcagttgaattctgtgacaactgaggacacagccacatattactgtgcaagatcgggatggttactaccctactggtatttcgatgtctggggcgcagggaccacggtcaccgtctcctca

SEQ ID NO: 6 - DNA, 324 bp, synthetic construct
  misc_feature 1..324: note = VL sequence
  source 1..324: mol_type = other DNA
gatgtccagataacccagtctccatcttatcttgctgcatctcctggagaaaccattactattaattgtagggcaagtaagagtatcagcaaatatttagcctggtatcaagagaagcctgggaaaactaatgagcttcttatctactctggatccactttgcaatctggaattccatcaaggttccgtggcagtggatctggtacagatttcactctcaccatcagtagcctggagcctgaagattttgcaatgtattactgtcaacagcacaatgaatacacgctcacgttcggtggtgggaccaagctggagctgaaacgg

SEQ ID NO: 7 - DNA, 324 bp, synthetic construct
  misc_feature 1..324: note = VL sequence
  source 1..324: mol_type = other DNA
gatgttcagatcacacagagcccaagctacctcgcagcatctccaggcgagactatcactatcaactgtagagcctccaagtccatttctaagtatctggcttggtatcaggagaagccaggtaagaccaacgagctgctgatctactctggaagcacactccagtccggtatcccatccagattcagaggttctggttctggcaccgatttcaccctgacaatcagctctctggagcctgaagatttcgcaatgtactattgtcagcagcataacgagtataccctgactttcggtggaggaacaaagctcgagctgaagcgt

SEQ ID NO: 8 - DNA, 363 bp, synthetic construct
  misc_feature 1..363: note = VH sequence
  source 1..363: mol_type = other DNA
gacgtgcagctccaagagagcggtcctggtctggtgaagccttctcaaagcctgtccctgacctgtaccgtttctggctatagcatcacttctgactacgcctggaactggattaggcagtttcctggtaacaagctggaatggatgggctacattagctacaccctgactacaggttacaatccctctctgaagtctaggatttctatcactcgcgactcctctaagaaccagttcttcctgcaactgaatagcgtgacaacagaagatactgctacatactactgtgcaagatccggttggctgctgccttactggtacttcgatgtgtggggtgccggcacaaccgtgactgtcagctcc

(19) United States
(12) Patent Application Publication
(10) Pub. No.: US 2024/0150443 A1
(43) Pub. Date: May 9, 2024

(54) ANTIVIRALS AGAINST MEASLES VIRUS

(71) Applicant: The Trustees of Columbia University in the City of New York, New York, NY (US)

(72) Inventors: Matteo POROTTO, New York, NY (US); Anne MOSCONA, New York, NY (US); Branka HORVAT, Lyon (FR); Cyrille MATHIEU, Lyon (FR)

(21) Appl. No.: 18/491,733

(22) Filed: Oct. 20, 2023

Related U.S. Application Data
(63) Continuation-in-part of application No. PCT/US22/25619, filed on Apr. 20, 2022.
(60) Provisional application No. 63/177,224, filed on Apr. 20, 2021.

(51) Int. Cl. C07K 16/10; A61K 39/00; A61P 31/14

(52) U.S. Cl. CPC C07K 16/1027; A61P 31/14; A61K 2039/505; C07K 2317/622; C07K 2317/76

(57) ABSTRACT

Compositions, and methods of preventing Measles and other viral infections using the compositions, related to single-chain variable fragment- (scFv-) based antivirals that bind to the measles virus (eV) F peptide and block F-mediated membrane fusion, thereby preventing infection. The scFv may interact synergistically with MeV fusion inhibitory peptides, such as HRC4. The scFv may be administered to the patient by airway, such as intranasally, and may be co-administered with one or more MeV fusion inhibitory peptides, such as HRC4.

### CROSS-REFERENCE TO RELATED APPLICATIONS

This application is a continuation-in-part of International Application No. PCT/US2022/025619, filed on Apr. 20, 2022, which claims benefit of U.S. Provisional Application No. 63/177,224 filed Apr. 20, 2021, the contents of which are hereby incorporated by reference.

All patents, patent applications and publications cited herein are hereby incorporated by reference in their entirety. The disclosures of these publications in their entireties are hereby incorporated by reference into this application.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### GOVERNMENT SUPPORT

This invention was made with government support under grants AI121349, NS091263, NS105699, and AI146980, awarded by the National Institutes of Health. The government has certain rights in the invention.

### INCORPORATION-BY-REFERENCE OF MATERIAL ELECTRONICALLY FILED

Incorporated by reference in its entirety herein is a computer-readable nucleotide/amino acid sequence listing submitted concurrently herewith and identified as follows: One 14,104-byte XML file named “SeqList_070.xml” created on Jan. 19, 2024.

### BACKGROUND OF THE INVENTION

Progress towards global measles virus (MeV) eradication has been halted and measles is a re-emerging disease, especially in the U. S. and in Europe where it was once considered to be eradicated. MeV infection can cause a profound transient immune suppression with depletion of humoral immunity in healthy individuals, and causes even more serious complications in immunocompromised individuals. Because an effective measles vaccine is available antiviral development for measles has not been prioritized, but recent outbreaks have highlighted the need for drugs to prevent transmission in unvaccinated populations and to protect and treat immunocompromised individuals.

Despite the availability of an effective measles virus (MeV) vaccine and efforts to increase vaccine coverage by the Measles & Rubella Initiative launched by the WHO, UNICEF, and their partners, MeV has not been eradicated and has caused 100,000-140,000 deaths globally every year since 2010¹⁻³. MeV eradication via vaccination is hindered by poor protection in the presence of maternal antibodies and a relatively low vaccination coverage, often related to concerns about vaccination safety⁴. Additionally, the current vaccine is a live-attenuated viral vaccine and cannot be used in severely immunosuppressed people. Thus, measles infection has been regularly resurfacing in Europe and the United States.

### SUMMARY OF THE INVENTION

In certain aspects, the invention provides a single-chain variable fragment (scFv) that binds to the MeV F peptide. The scFv blocks F-mediated membrane fusion and spread of the MeV. In some embodiments, the scFv interacts synergistically with the MeV fusion inhibitory peptides. In some embodiments, the MeV fusion inhibitory peptide is HRC4.

In certain aspects, the invention provides a method of preventing measles in a subject in need thereof, which includes administering to the subject a scFv as described above. In some embodiments, the scFv is co-administered with the MeV fusion inhibitory peptide HRC4. In some embodiments, the scFv is administered with one or more pharmaceutically acceptable excipients.

In some embodiments, the scFv is administered per airway. In some embodiments, the scFv is administered intranasally. In some embodiments, the scFv is administered subcutaneously.

### BRIEF DESCRIPTION OF FIGURES

FIG. 1: mAb 186 antibody recognition of prefusion F is abrogated by a single amino acid change. (A) Differences in IC323 and G594 F proteins. (B-E) HEK293T cells transfected with either pCAGGS-MeV IC323 F (B) or pCAGGS-MeV G954 F (C) were stained using anti-F mAb 186 24 h after transfection. (D-E) HEK293T cells transfected with either pCAGGS-MeV IC323 F R70K (D) or pCAGGS-MeV G954 F K70R (E) were stained using anti-F mAb 186 24 h post-transfection at 37° C. (F) mAb 186 staining was quantified as relative luminescent units (RLU). mAb 77.4 inhibits MeV IC323 infection: (H-K) VeroSLAM cells were infected with MeV IC323-EGFP at a multiplicity of infection (MOI)=0.001. After 6 hours, cells were (H) untreated (I) treated with serial dilutions of supernatant fluids from hybridomas producing anti-F mAb 77.4 (J) mAb Y503 (K) MeV infection in Vero-SLAM cells either untreated (NT) or treated at the indicated dilution of supernatant fluids from hybridomas producing anti F-mAb 77.4 or mAb Y503 were quantified using fluorescent spectroscopy. Data are expressed as relative fluorescence units (RFUs). Each bar represents the mean (±standard error) of results from at least 3 separate experiments.

FIG. 2: mAb 77.4 recognizes both MeV G954 and IC323 pre-fusion F. HEK293T cells transfected with (A) pCAGGS-empty vector; (B) pCAGGS-MeV IC323 F; (C) pCAGGS-MeV G954 F; (D) pCAGGS-MeV IC323 F R70K; or (E) pCAGGS-MeV G954 F K70R. (F) HEK293T cells were transfected with pCAGGS-MeV IC323 F and incubated 24 h at 32° C. (G) Quantification of positive staining.

FIG. 3: Single-chain fragment (scFv) derived from mAb 77.4 inhibits MeV infection in vitro. (A) Sequence of the single-chain variable fragment antibody derived from anti-MeV F mAb 77.4. (B) scFv inhibits MeV infection: (D) Fusion of MeV G954 or IC323 H/F co-expressing cells with SLAM-bearing cells in the presence of scFv. VH region is SEQ ID NO:1; VL region is SEQ ID NO:2; entire scFv shown is SEQ ID NO:3.

FIG. 4: mAb 77.4-derived scFv prevents MeV infection in vivo in cotton rats.

FIG. 5: Synergistic activity of mAb 77.4-derived scFv and HRC4 inhibitor in fusion assays. (A) Dose response of scFv on fusion inhibition. (B) Dose response of HRC4 fusion inhibition. (C) The theoretical responses at which additivity would occur based on the dose-response curves in A and B. (D) Plot of the additive response observed in the presence of each drug combination. (E) Loewe Synergy and antagonism surface of scFv and HRC4 at indicated concentrations. (F) Multicolor surface of the synergism response values in comparison to the additive outcome. Data are from three independent experiments.

FIG. 6: Mutating MeV IC323 F and MeV G954 F at amino acid 70 did not affect surface level expression. (A) western blotting; (B) quantitated with image J.

FIG. 7: Synergistic activity of mAb 77.4 and HRC4 inhibitor in fusion assays. (A) Dose response of mAb 77.4 on fusion inhibition. (B) Dose response of HRC4 on fusion inhibition. (C) The theoretical responses at which additivity would occur based on the dose-response curves in A) and B). (D) Plot of the additive response observed in the presence of each drug combination. (E) Loewe Synergy and antagonism surface of mAb 77.4 and HRC4 at indicated concentrations. (F) Multicolour surface of the synergism response values in comparison to the additive outcome.

FIG. 8: Depiction of GS66077-1 pTRIOZ-hIgG1_HRV3C_77.1, 77.1 mAb with variable domains VL and VH cloned in pTRIOZ-hIgG1 vector.

FIG. 9: Plots comparing inhibitory efficacy of human monoclonal antibody (77.1 mAb) on the areas of plaques formed by the indicated recombinant MeV viruses with eGFP in infected Vero SLAM (FIG. 9A) or Vero cells (FIG. 9B).

FIG. 10: Plot comparing antiviral prophylactic efficacy for MeV in vivo in cotton rats, after the 77.1 mAb was administrated by subcutaneous delivery, 12 hours before infection, of either the indicated amount of mAb 77.1 (0.1 mg/Kg or 1 mg/Kg) or vehicle as control, reported as TCID₅₀/g of lung tissue.

FIG. 11: Plot comparing antiviral prophylactic efficacy for MeV in vivo in cotton rats, after the rats were infected intranasally with 10⁵ TCID₅₀/ml MeV B3-eGFP wild type. Animals received either the indicated amount of the mAb 77.1 (0.1 mg/kg or 1 mg/kg) or vehicle subcutaneously 12 hours prior to infection or 24 hours post infection (1 mg/kg). Four days after infection, the animals were euthanized and the viral titers in the animals' lungs were quantified. Treatment with 77.1 mAb reduced the viral titer in the lungs compared with the untreated control (FIG. 11).

### DETAILED DESCRIPTION OF THE INVENTION

MeV infects activated SLAM/CD150-expressing immune cells in the respiratory tract, allowing the virus to invade the immune system⁵. Once it has reached the draining lymph nodes, the virus proliferates in SLAM/CD150-expressing lymphocytes and then proceeds to cause viremia. Late in infection, MeV infects respiratory epithelial cells via nectin-4 expressed on the basolateral membranes of these cells; from this location, MeV exits the host respiratory tract to permit transmission^{6,7}. Upon initial attachment of MeV to cell surface receptors, entry is mediated by the concerted actions of the MeV receptor-binding (H) and fusion (F) proteins, which comprise the H/F viral fusion complex on the surface of the virus^{5,8,9}. Infected cells synthesize F as a precursor (F0) that is cleaved within the cell to yield the pre-fusion F complex comprising three C-terminal F1 subunits, associated via disulfide bonds with three N-terminal F2 subunits. Newly produced viral particles display this trimeric F structure kinetically trapped in a metastable conformation, in complex with H on the outer surface of the viral membrane¹⁰. F is primed for fusion activation upon engagement of the H glycoprotein by an entry receptor (i.e., SLAM or nectin-4 for wild-type strains) expressed on the surfaces of target cells^{6,7}. After receptor engagement, H triggers the pre-fusion F protein to undergo a structural rearrangement leading to the extension of F, allowing F to insert its hydrophobic fusion peptide into the target host cell membrane. F then refolds into a stable post-fusion 6-helix bundle structure, bringing the viral and target cell membranes together to initiate the formation of the fusion pore.

Viral infection can be blocked at several steps during entry. Monoclonal antibodies that block H-receptor interaction and thereby interfere with entry have been described¹¹. Small molecules that prevent F activation have been also identified¹²⁻¹⁴. Entry can also be inhibited by interfering with the refolding step of F protein to attain a stable post-fusion state—the process that drives membrane fusion during viral entry. This step relies on the interaction between the complementary heptad repeat (HR) regions localized at the N- and C-termini of the protein (HRN and HRC respectively), and can be inhibited by F peptides derived from the HR regions¹⁵. In previous studies, the potential of HR-targeting peptides as antiviral agents was demonstrated in vivo^{11,16,17}. It was also shown that combining anti-H antibodies with HR antiviral peptides greatly increases antiviral efficacy¹¹. In this work, we assessed viral neutralization by three mouse monoclonal antibodies that specifically target the F protein in its pre-fusion state. From the most potent antibody we engineered a single-chain fragment (scFv), and we show that it retains fusion inhibitory and virus neutralizing activity in vitro and in vivo. The inhibition of infection in vivo after intranasal administration of scFv shows that targeting the pre-fusion state of MeV F protein is an anti-MeV strategy.

In certain aspects, the invention provides a single-chain variable fragment (scFv) that binds to the MeV F peptide. The scFv blocks F-mediated membrane fusion and spread of the MeV. In some embodiments, the scFv interacts synergistically with the MeV fusion inhibitory peptides. In some embodiments, the MeV fusion inhibitory peptide is HRC4.

In certain aspects, the invention provides a method of preventing or treating measles in a subject in need thereof, which includes administering to the subject a scFv as described above. In some embodiments, the scFv is co-administered with the MeV fusion inhibitory peptide HRC4. In some embodiments, the scFv is administered with one or more pharmaceutically acceptable excipients. In some embodiments, the scFv comprises SEQ ID NO:1 and/or SEQ ID NO:2. In some embodiments, the scFv comprises SEQ ID NO:3. In some embodiments, the MeV fusion inhibitory peptide HRC4 comprises SEQ ID NO:4:
(Ac-Pro-Pro-Ile-Ser-Leu-Glu-Arg-Leu-Asp-Val-Gly-Thr-Asn-Leu-Gly-Asn-Ala-Ile-Ala-Lys-Leu-Glu-Asp-Ala-Lys-Glu-Leu-Leu-Glu-Ser-Ser-Asp-Gln-Ile-Leu-Arg-Gly-Ser-Gly-Ser-Gly-Cys-PEG4_bisMaleimide)₂-Cholesterol

In some embodiments, preventing measles encompasses reducing the extent of or reducing the development of a measles infection in a previously non-infected subject. In some embodiments, treating measles means reducing one or more symptoms of an extant measles infection in a subject. In preferred embodiments, the subject is a human.

In some embodiments, the scFv is administered per airway. In some embodiments, the scFv is administered intranasally. In some embodiments, the scFv is administered subcutaneously.

In some embodiments, the scFv (derived from antibody specific for the virus of interest) is administered to treat or prevent a viral infection other than measles. A scFv may be administered, for example, to treat or prevent an infection from a Nipah virus, Human parainfluenza virus, or a Respiratory Syncytial Virus (RSV). The administration may be through any route appropriate for the formulation, such as intranasally or subcutaneously. Similarly, the preparation may be formulated for administration orally, parenterally, sublingually, topically, or by inhalation. Parenteral administration may be, for example, intramuscular, intravenous, subcutaneous, or intradermal.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1: Neutralizing Activity of Antibodies that Target the Pre-Fusion Conformation of the MeV Fusion Protein

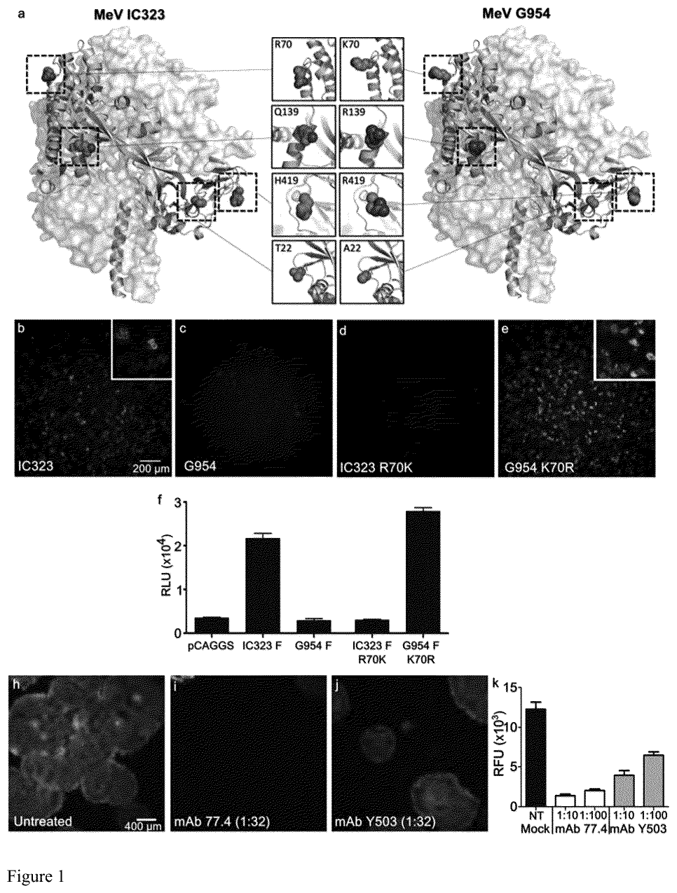

To examine the binding and neutralizing properties of several anti-MeV monoclonal antibodies (mAbs), we used MeV strains IC323 F¹⁸ and MeV G954 (wild type circulating strains). FIG. 1A shows the differences between MeV IC323 and MeV G594 in a schematic depiction of one MeV F monomer in its pre-fusion state (PDBID: 5YXW). In previous work, several mAbs with neutralizing activity against MeV were identified^{19,20} and the neutralization properties of one of these mAbs (mAb 186) correlated well with its ability to recognize the F protein in its pre-fusion state²¹. We assessed the binding of mAb 186 to pre-fusion F proteins from the two MeV strains, MeV IC323 F (FIG. 1B), and MeV G954 (FIG. 1C). Immunofluorescent staining of F-expressing cells with mAb 186 showed that mAb 186 recognized MeV IC323 F (FIG. 1B) but did not recognize MeV G954F (FIG. 1C). Quantification of staining was performed using our established methods^{18,22} with results depicted as relative luminescent units (RLU) (FIG. 1F).

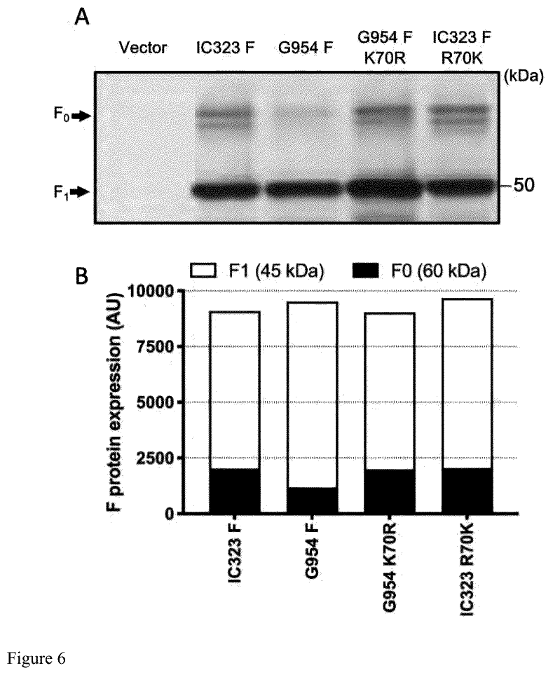

MeV IC323 and MeV G954 F differ at four residues: T22A, R70K, Q139R, and H419R, residues all found in solvent exposed regions (FIG. 1A). The boxes contain close-up comparisons of these mutations with their carbon (magenta), nitrogen (blue) and oxygen (orange) atoms depicted as spheres (FIG. 1A). Each of these amino acid alterations were separately introduced into both strains of MeV. Cells were transfected with pCAGGS expressing MeV IC323 F bearing the R70K mutation (FIG. 1D), and pCAGGS expressing MeV G954 F bearing the K70R mutation (FIG. 1E). Quantification of staining is depicted as relative luminescent units (RLU) (FIG. 1F). The R70K mutation was responsible for the lack of recognition of MeV G954 pre-fusion F by mAb 186 (FIG. 1 D,F). Cells transfected with G954 F with the K70R mutation (FIG. 1 E, F) show mAb 186 recognition. FIG. 6 shows a representative cell surface biotinylation pulldown, demonstrating that the difference in cell surface recognition by mAb 186 was not due to differential protein expression.

In an effort to identify a more broadly reactive mAb, the neutralizing activity of two other previously identified mAbs, mAb 77.4 and mAb Y503, was assessed in the experiments shown in FIG. 1 I-K. Vero cells expressing the measles receptor SLAM (Vero-SLAM) were infected with MeV IC323-EGFP virus (the expression of fluorescent protein permits tracking of the spread of infection). Six hours post-infection, the cells were treated with serial dilutions of mAbs 77.4 or Y503. Infection was monitored over time and 5 days after infection, immunofluorescence was measured to quantify viral spread (FIG. 1H-J) as previously described²³ (FIG. 1K). Both mAbs inhibit viral spread, with mAb 77.4 being more potent than Y503; we therefore proceeded to determine which conformational state of F is recognized by mAb 77.4 and whether this mAb can recognize the MeV G954 strain.

### Example 1 Methods—See FIG. 1

### mAb 186 Antibody Recognition of Prefusion F is Abrogated by a Single Amino Acid Change

(A) Differences in IC323 and G594 measles virus (MeV) fusion (F) protein from wild type (wt) strains were modeled in PyMOL (Schrödinger) against the crystal structure of MeV prefusion F ectodomain (PDBID: 5YXW). The sculpting feature in PyMOL, with a 6.0 Å radius, was used to minimize steric clashes introduced by the mutations. (B-E) HEK293T cells transfected with either pCAGGS-MeV IC323 F (B) or pCAGGS-MeV G954 F (C) were stained using anti-F mAb 186 24 h after transfection.

(D-E) HEK293T cells transfected with either pCAGGS-MeV IC323 F R70K (D) or pCAGGS-MeV G954 F K70R (E) were stained using anti-F mAb 186 24 h post-transfection at 37° C. (F) mAb 186 staining of MeV IC323 F,G954 F MeV IC323 F R70K or G954 F K70R was quantified as relative luminescent units (RLU). Each bar represents the mean (±standard error) of results from at least 3 separate experiments.

### mAb 77.4 Inhibits MeV IC323 Infection

(H-K) VeroSLAM cells were infected with MeV IC323-EGFP at a multiplicity of infection (MOI)=0.001. After 6 hours, cells were either untreated (H) or treated with serial dilutions of supernatant fluids from hybridomas producing anti-F mAb 77.4 (I) or mAb Y503 (J). Immunofluorescence was performed 5 days after infection (scale bar=200 μm). (K) MeV infection in Vero-SLAM cells either untreated (NT) or treated at the indicated dilution of supernatant fluids from hybridomas producing anti F-mAb 77.4 or mAb Y503 were quantified using fluorescent spectroscopy. Data are expressed as relative fluorescence units (RFUs). Each bar represents the mean (±standard error) of results from at least 3 separate experiments.

### Example 2: mAb 77.4 Recognizes the IC323 and G954 F Proteins in their Pre-Fusion State

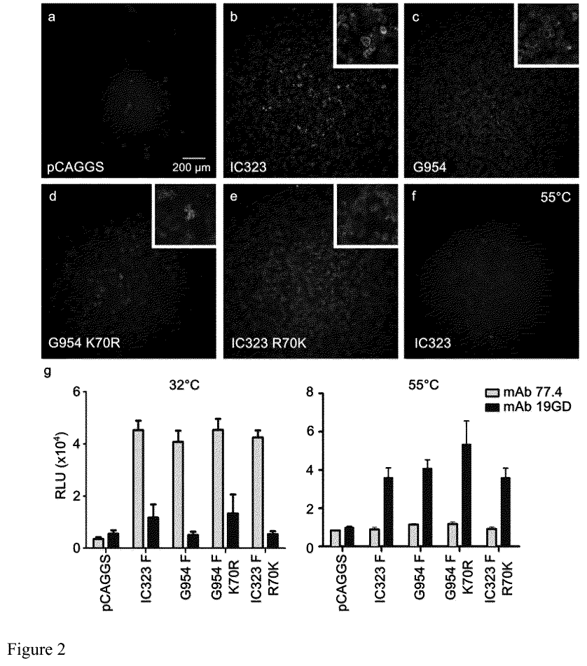

To assess whether the neutralization activity of mAb 77.4, like that of mAb 186, correlates with its ability to recognize the F protein in its pre-fusion state we carried out immunofluorescent staining of F-expressing cells with mAb 77.4, using MeV IC323 F, MeV G954 F, MeV IC323 F bearing the R70K mutation, and MeV G954 F bearing the K70R mutation (FIG. 2). The mAb 77.4 recognized all the wt and mutant MeV F proteins in their pre-fusion state (FIG. 2 A-E), but not in their post-fusion states induced by heat (FIG. 2F). For this experiment, cells expressing vector alone (FIG. 2A), MeV IC323 F (FIG. 2 B-F), MeV G954 F (FIG. 2C), MeV G954 F bearing the K70R mutation (FIG. 2D), or MeV IC323 F bearing the R70K mutation (FIG. 2E) were then incubated for 60 minutes at 32° C. (FIG. 2A-E) or 55° C. (FIG. 2F) and stained with mAb 77.4 (FIG. 2 A-F). The 55° C. incubation is sufficient to shift pre-fusion F into its post-triggered state^{18,21}. As expected, mAb 77.4 failed to recognize MeV IC323 F after exposure to high temperature which exposes the post-triggered F (FIG. 2F). FIG. 2G depicts quantification of staining as relative luminescence units (RLU). To confirm the pre- vs. post-triggered state of F we also quantified the amount of post-fusion F with mAb 19GD that recognizes post-fusion F^{18,21,24} (FIG. 2G). The mAb 77.4 thus recognizes all the MeV F proteins in their pre-fusion state (at 32° C.) but not once triggered (55° C.) and has a broader recognition spectrum than mAb 186 (compare FIG. 1 to FIG. 2). We therefore selected mAb 77.4 for further engineering.

### Example 2 Methods—See FIG. 2

### mAb 77.4 Recognizes Both MeV G954 and IC323 Pre-Fusion F

(A-E) HEK293T cells transfected with either pCAGGS-empty vector (A); pCAGGS-MeV IC323 F (B); pCAGGS-MeV G954 F (C); pCAGGS-MeV IC323 F R70K (D); or pCAGGS-MeV G954 F K70R (E) were stained using anti-F mAb 77.4 after 24 h at 32° C. (F) HEK293T cells were transfected with pCAGGS-MeV IC323 F and incubated 24 h at 32° C. Twenty-four hours later the cells were incubated for 1 h at 55° C. and stained using anti-F mAb 77.4. (G) Quantification of positive staining in HEK293T cells transfected with the indicated vectors after incubation for 1 h at 32° C. or 55° C. and staining with either anti-F mAb 77.4 or anti-post-fusion F mAb, 19GD. Staining was quantified as RLU. Each bar represents the mean (standard error) of results from at least 3 separate experiments.

### Example 3: Single-Chain Fragment Variable (scFv) from mAb 77.4 Maintains F Recognition and Neutralization Properties In Vitro

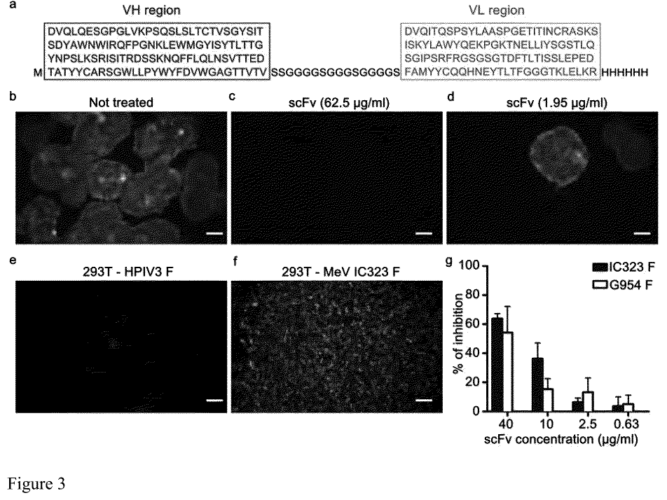

A single-chain fragment (scFv) was derived by fusing the variable regions of the heavy (VH) and light chains (VL) of mAb 77.4 (FIG. 3A). The scFv was expressed in eukaryotic cells and purified by affinity purification using a 6×-hisitidine tag (see Materials and Methods). The neutralizing activity of the scFv was assessed in the experiments shown in FIG. 3B. Vero-SLAM cells were infected with MeV IC323-EGFP virus and six hours post-infection, the cells were treated with serial dilutions of the scFv (62.5 μg/ml and 1.95 μg/ml are shown in 3C and CD) and immunofluorescent syncytium formation was measured to quantify viral spread 48 hours post-infection. The scFv clearly inhibited viral infection (FIG. 3B).

To assess the specificity of the scFV derived from mAb 77.4 for MeV F, cells expressing either MeV IC323 F or the human parainfluenza virus 3 fusion protein (HPIV3 F) were incubated with the scFv and subjected to immunofluorescence using a fluorescently-labeled Alexa 488 anti 6-histidine antibody (FIG. 3E-F). Only the cells expressing MeV IC323 F, not those expressing HPIV3 F, were recognized by the scFV (FIG. 3E-F). Finally, we assessed whether the scFv blocked cell-cell fusion mediated by either MeV IC323 or G954 H/F fusion complexes (FIG. 3G). For this experiment we used a beta-galactosidase (β-Gal) complementation assay¹⁸ that measures the fusion of cells that express viral envelope glycoproteins (MeV IC323 or G954 H/F) with cells that express the MeV receptor SLAM (FIG. 3G). HEK293T (“target cells”) were transiently transfected with SLAM and the omega reporter subunit of beta-galactosidase, and incubated for the indicated time period in the presence of different concentrations of inhibitors with “effector cells” co-expressing MeV G954 or IC323 H and F together with the alpha reporter subunit of beta-galactosidase. Fusion between the target and effector cells leads to reconstitution of beta-galactosidase activity, which we quantified using the luminescence-based Galacto-Star™ β-Galactosidase Reporter Gene kit (ThermoFisher, US). The scFv inhibited cell fusion mediated by both the IC323 and G954 H/F fusion complexes (FIG. 3G).

### Example 3 Methods—See FIG. 3

### Single-Chain Fragment (scFv) Derived from mAb 77.4 Inhibits MeV Infection In Vitro

(A) Sequence of the single-chain variable fragment antibody derived from anti-MeV F mAb 77.4. (B) scFv inhibits MeV infection: Vero-SLAM cells either treated at the indicated concentration of scFv or not treated were infected with MeV IC323-EGFP at MOI=0.001. Images were obtained using epifluorescent microscopy 48 h post-infection (scale bar=200 μm). (C) HEK293T cells were transfected with either pCAGGS-HPIV3 F or MeV IC323 F and stained using scFv 24 h post-transfection (scale bar=200 μm). (D) Fusion of MeV G954 or IC323 H/F co-expressing cells with SLAM-bearing cells in the presence of scFv at indicated concentrations was quantified at 6 h using a β-galactosidase complementation assay. Results are presented as percent reduction in luminescence (Y-axis), compared to no treatment. Each bar represents the mean (±standard error) of results from at least 3 separate experiments.

### Example 4: In Vivo Prophylaxis Against MeV Infection Using scFv Construct Derived from mAb 77.4

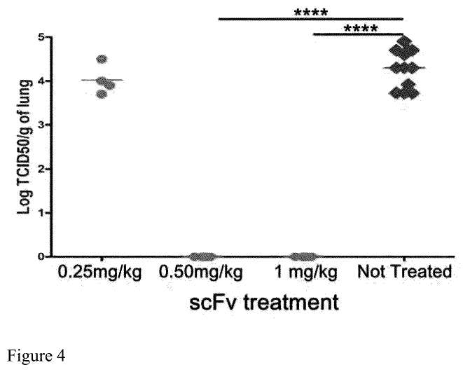

To assess anti-MeV prophylaxis by the scFv in vivo, we used cotton rats (CR) as an infection model. The scFv antibody construct was administrated via intranasal delivery 24 and 12 h before infection. Four days after infection, the animals were euthanized, and the viral titers in the animals' lungs were quantified. Treatment with scFv significantly reduced viral titers in the lungs, compared with untreated controls (FIG. 4). We tested three doses (0.25, 0.5 and 1 mg/ml) and observed that the two highest doses of scFv completely inhibited lung infection.

### Example 4 Methods—See FIG. 4

### mAb 77.4-Derived scFv Prevents MeV Infection In Vivo in Cotton Rats

Cotton rats were infected intranasally with 10⁵ TCID50/ml of MeV WTFb. Animals received either the indicated amount of mAb 77.4-derived scFv construct (n=4) or vehicle (n=8) 24 h and 12 h prior to infection. Animals were euthanized 5 days after infection. Lungs were collected and homogenized for TCID50 titration. Results are presented as TCID50/g of lung. Statistical analyses were performed using the Mann-Whitney U-test.

### Example 5: Combination of scFv and HRC4 Antiviral Peptide is Synergistic Vs. MeV

Since the scFv construct is derived from an antibody that targets the pre-fusion state of F, we investigated the possibility of synergy with the HR-targeting antiviral peptides (HRC4) that target the refolding step of the F protein after activation.

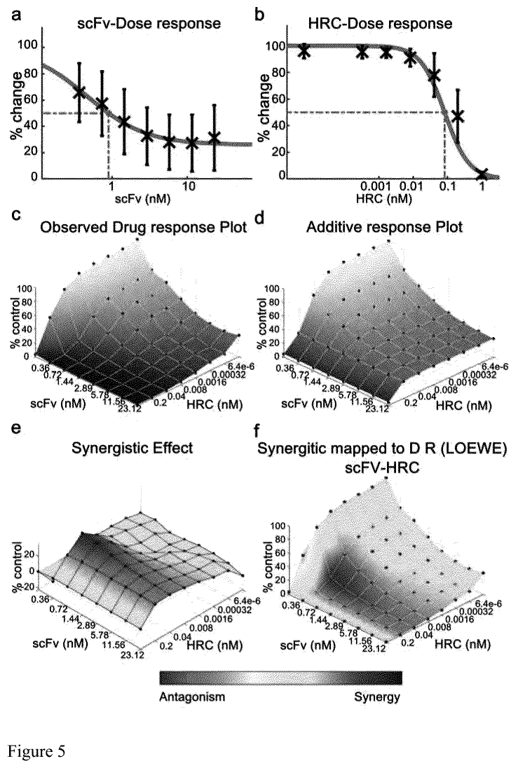

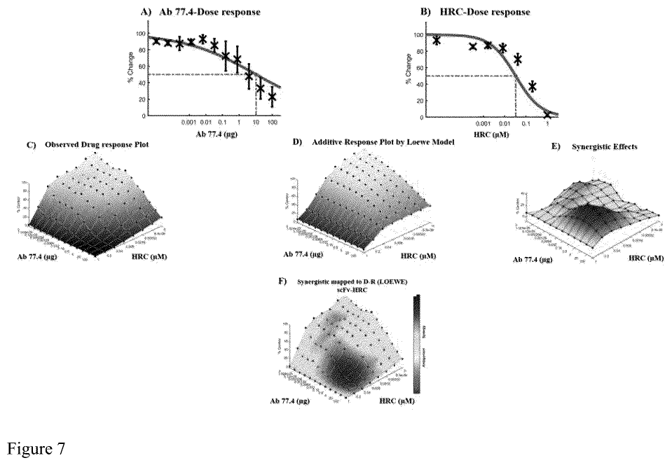

For the experiment in FIG. 5, HEK293T “target” cells were transiently transfected with Nectin-4 and the omega reporter subunit of beta-galactosidase, and incubated for the indicated time periods in the presence of different concentrations of scFv and HRC4 with “effector” cells co-expressing viral glycoproteins (H and F) and the alpha reporter subunit of beta-galactosidase. Fusion between the target and effector cells was quantified as for FIG. 3G. The results were analyzed with Combenefit software to obtain the additive curve from the actual curves of the two inhibitors alone, and to compare it to the actual curves of the inhibitors added together. There is significant synergism between scFv and HRC4 for inhibiting viral fusion. Similar results were obtained when we assessed the combined effects of mAb 77.4 and HRC4 on viral fusion (see FIG. 7).

### Example 5 Methods—See FIG. 5

### Synergistic Activity of mAb 77.4-Derived scFv and HRC4 Inhibitor in Fusion Assays

A beta-galactosidase complementation-based assay was performed to assess the effects of mAb 77.4-derived scFv and the HRC4 inhibitor on viral fusion. The data were analyzed using Combenefit software. (A) Dose response of scFv on fusion inhibition. (B) Dose response of HRC4 fusion inhibition. (C) The theoretical responses at which additivity would occur based on the dose-response curves in A and B. (D) Plot of the additive response observed in the presence of each drug combination. (E) Loewe Synergy and antagonism surface of scFv and HRC4 at indicated concentrations. (F) Multicolor surface of the synergism response values in comparison to the additive outcome. Data are from three independent experiments.

### Example 6: Mutating MeV IC323 F and MeV G954 F at Amino Acid 70 Did not Affect Surface Level Expression. See FIG. 6

Expression of the different MeV F proteins presented in FIG. 1 was assessed using cell-surface biotinylation pull-down followed by western blotting (A) and quantitated with image J (B).

Ongoing MeV outbreaks²⁵⁻³⁶ along with increased awareness of the compromised humoral response following wild-type MeV infection³⁴ point to the risks of measles as a re-emerging disease. In addition to the profound immune suppression observed in healthy individuals, measles infection can cause complications in the central nervous system (CNS), and is life-threatening for immunocompromised individuals. The estimated annual deaths from measles increased from 89,780 in 2016 to 207,500 in 2019³⁷. During the SARS-CoV-2 pandemic, the incidence of other respiratory infections was reduced, likely due to the precautions aimed at preventing respiratory pathogen transmission. However, the pandemic also led to reduced childhood measles vaccination and the specter of larger measles outbreaks in the future³⁷.

We found that a single-chain variable fragment (scFv) construct derived from an IgG monoclonal antibody (mAb 77.4) (lacking the constant region) binds to MeV F and blocks both F-mediated membrane fusion and viral spread through cultured cells. The combination of fusion inhibitory peptides (HRC4) and scFv was synergistic. The scFv interacts with the pre-fusion conformation of F, preventing activation of the fusion processes, while the HRC4 peptide blocks the subsequent refolding of F into its post-fusion state that drives viral entry.

The in vitro potency of the scFv construct is less than that of the full antibody from which it was derived, possibly due to loss of avidity or affinity for the antigen. Increasing affinity maturation could increase the antiviral potency of the scFv. Grafting the variable region to a single-chain antibody would also reduce the size of the inhibitor and may permit the construct to cross the blood-brain barrier in the event of CNS manifestations of disease. Intranasal administration of the scFv construct lacking the immunoglobulin constant region was sufficient to neutralize MeV infection, suggesting that these modifications may be tolerated without loss of efficacy.

It has been shown that for a different previously-identified mAb to MeV F (mAb 186) an escape mutation readily arose, and this escape mutation was present in a naturally occurring wild-type isolate, MeV G954 (see FIG. 1). Of the four residues where MeV G954 differs from MeV IC323, the mutation that conferred escape to mAb 186 is residue 70 (FIG. 1), denoted as residue 73 in²⁰. Our results indicate that mAb 77.4 has broader antiviral activity than mAb 186, but the possibility of eventual resistance to mAb 77.4 and its scFv construct will be assessed in future studies.

To prevent transmission of respiratory viruses, targeting viral entry is an ideal approach³⁸. Future strategies leveraging the results shown here will entail either combination of the two entry inhibitors, or conjugation of HRC4 and scFv (or a single-chain antibody) into a bifunctional single inhibitor. A dual-function entry inhibitor based on protein-protein interactions at distinct sites may reduce the potential for emergence of escape variants.³⁹

### Example 7: Synergistic Activity of mAb 77.4 and HRC4 Inhibitor in Fusion Assays. See FIG. 7

A beta-galactosidase complementation-based assay was performed to assess the effects of mAb 77.4-derived scFv and HRC4 inhibitor on viral fusion. Data were analyzed using Combenefit software. (A) Dose response of mAb 77.4 on fusion inhibition. (B) Dose response of HRC4 on fusion inhibition. (C) The theoretical responses at which additivity would occur based on the dose-response curves in A) and B). (D) Plot of the additive response observed in the presence of each drug combination. (E) Loewe Synergy and antagonism surface of mAb 77.4 and HRC4 at indicated concentrations. (F) Multicolour surface of the synergism response values in comparison to the additive outcome. Data are from three independent experiments.

### Example 7 Material and Methods

Peptides and chemicals. MeV F-derived fusion inhibitory peptides have been previously described^{16,17}. Briefly, 36-aa peptides derived from the C-terminal heptad repeat region of MeV F protein were synthesized. Dimeric cholesterol-conjugated (HRC4) forms of the peptides were used in this study.

Hybridoma sequencing (Creative Biolabs, CBL). TRIZol lysates of one hybridoma cell line were sequenced. VH and VK region sequences are listed below.

VH = DNA
(SEQ ID NO: 5)
GATGTGCAGCTTCAGGAGTCGGGACCTGGCCTGGTGAAACCTTCTCAGT
CTCTGTCCCTCACCTGCACTGTCTCTGGCTACTCAATCACCAGTGATTA
TGCCTGGAACTGGATCCGGCAGTTTCCAGGAAACAAACTGGAGTGGATG
GGCTACATAAGCTACACTCTTACCACTGGCTACAACCCATCTCTCAAAA
GTCGAATCTCTATCACTCGAGACTCATCCAAGAACCAGTTCTTCCTGCA
GTTGAATTCTGTGACAACTGAGGACACAGCCACATATTACTGTGCAAGA
TCGGGATGGTTACTACCCTACTGGTATTTCGATGTCTGGGGCGCAGGGA
CCACGGTCACCGTCTCCTCA
VK = DNA
(SEQ ID NO: 6)
GATGTCCAGATAACCCAGTCTCCATCTTATCTTGCTGCATCTCCTGGAG
AAACCATTACTATTAATTGTAGGGCAAGTAAGAGTATCAGCAAATATTT
AGCCTGGTATCAAGAGAAGCCTGGGAAAACTAATGAGCTTCTTATCTAC
TCTGGATCCACTTTGCAATCTGGAATTCCATCAAGGTTCCGTGGCAGTG
GATCTGGTACAGATTTCACTCTCACCATCAGTAGCCTGGAGCCTGAAGA
TTTTGCAATGTATTACTGTCAACAGCACAATGAATACACGCTCACGTTC
GGTGGTGGGACCAAGCTGGAGCTGAAACGG

Cell cultures. HEK293T (human kidney epithelial), Vero (African green monkey kidney) and Vero-SLAM/CD150 cells were grown in Dulbecco's modified Eagle's medium (DMEM; Life Technologies; Thermo Fisher Scientific) supplemented with 10% fetal bovine serum (FBS, Life Technologies; Thermo Fisher Scientific) and antibiotics at 37° C. in 5% CO2. The Vero-SLAM/CD150 culture media was supplemented with 1 mg/ml Geneticin (Thermo Fisher Scientific).

Plasmids. The genes of MeV G954 and IC323 F and H proteins (wild-type and mutants), as well as CD150/SLAM and nectin-4 were codon optimized, synthesized, and subcloned into the mammalian expression vector pCAGGS between EcoRI and BglII sites by Epoch Biolabs (Missouri City, TX).

Transient expression of viral glycoproteins. Transfections were performed in HEK293T cells according to the protocols of the Lipofectamine 2000 manufacturer (Invitrogen). Alternatively, cells were transfected using polyethyleneimine (PEI; Polyscience Inc) Briefly, DNA constructs, dissolved in Opti-MEM (Thermo Scientific), were mixed with PEI (1:2.5 ratio), and incubated for 20 min at room temperature and then added to the cells. After 4 h, the transfection mix was replaced with DMEM, supplemented with 10% FBS and antibiotics, and cells were incubated to allow protein expression overnight.

Recombinant scFv Expression and Purification (Creative Biolabs, CBL). Expression vector construction: the gene for the scFv antibody fragment was constructed by ligating the gene fragments encoding the heavy chain variable region (VH) and light chain variable region (VL) of the full-length antibody via a G4S linker. The scFv gene was synthesized using PCR-based methods and cloned into the pET21 vector for expression in a CBL-proprietary mammalian cell expression vector for protein expression in the HEK293F cell line. Plasmid amplification: the expression vectors were amplified in DH5a competent cells and extracted using plasmid extraction kits (QIAGEN). The extracted scFv expression vectors were then assayed for their A260/A280 ratios, endotoxin levels, and sterility before cell transfection. Expression cell line preparation: The HEK293F cell line was revived and cultured, until the cells reached desirable density and viability: log growth phase (˜1.8-2.2×10⁶ cell/ml). The HEK293F cell line was then transfected with the expression vector using PEI (Creative Biolabs, CBL). The cells were cultured for 4 days at 37° C. before protein purification. ScFv purification: 5-6 days post-transfection, the culture supernatant was harvested via centrifugation in the presence of a protease inhibitor, and the target protein was purified using a Ni-affinity column. Protein characterization: the stability of the scFv was tested using freeze-thaw experiments. The concentration of the scFv was determined using the Pierce™ BCA Protein Assay Kit, while the purity of the protein was analyzed using SDS-PAGE.

Immunofluorescence using F-conformation-specific mAbs. HEK293T cells transiently transfected with viral glycoprotein constructs were incubated overnight at 37° C. in complete medium (DMEM, 10% FBS). 20 h post-transfection, cells were transferred to the indicated temperatures for the times indicated in the figures. Thereafter, cells were incubated with mouse monoclonal antibodies (mAbs) that specifically detect MeV F in its pre-fusion conformation (1:1,000) for 1 h on ice. Cells were washed with PBS and incubated for 1 h on ice with Alexa-488 anti-mouse secondary antibody (1:500; Life Technologies), washed with PBS, and fixed for 10 min on ice with 4% paraformaldehyde (PFA), followed by incubation with a 1:1,000 dilution of DAPI (4′,6-diamidino-2-phenylindole; Thermo Fisher) in PBS for 60 min. Plates were washed; 0.01% sodium azide was added; and plates were imaged using an IN Cell Analyzer. Percentages of antibody-bound cells were determined using Cell Profiler software.

β-Gal complementation-based fusion assay. In order to quantify cell-to-cell fusion, we used a fusion assay based on alpha-omega complementation of β-galactosidase (β-Gal), which has been previously described^{40,41}. Briefly, 293T cells transiently transfected with the omega reporter subunit and the receptor plasmids were incubated with cells co-expressing viral glycoproteins (as indicated) and the alpha reporter subunit⁴². Cell fusion, which leads to β-Gal complementation, was stopped by lysing cells, and luminescence was obtained by adding the Galacton-Star substrate (Applied Biosystem) and measured using an nfinite M1000PRO (Tecan) microplate reader.

β-Gal assay for assessing F surface state using F-conformation-specific mAbs. HEK293T transiently transfected with viral glycoproteins constructs (as indicated) were incubated overnight at 32° C. with complete medium (DMEM, 10% FBS) and processed as previously described 18. Briefly, 20 h post-transfection, cells were transferred to the temperatures and times indicated in the figures. Thereafter, cells were incubated with mouse monoclonal antibodies (mAbs) that specifically detect MeV F in pre- or post-fusion conformation (1:1,000) for 1 h on ice. Cells were washed with PBS and then incubated for 1 h on ice with anti-mouse secondary antibody biotin-conjugated (1:500; Life Technologies). Next, cells were washed again with PBS and then fixed for 10 min on ice with 4% paraformaldehyde (PFA). Following fixation, cells were washed twice with PBS, blocked for 20 min on ice with 3% bovine serum albumin (BSA) in PBS, washed again, and then incubated for 1 h on ice with streptavidin conjugated to β-galactosidase (1:1,000, Life Technologies). Cells were washed with PBS, and the β-galactosidase substrate (1:50, Applied Biosystems) was added. Luminescence was measured using an Infinite M1000PRO (Tecan) microplate reader.

Cell surface expression using biotinylated protein pull-down. Cells expressing glycoproteins were incubated for 1 h with cyclohexamide to synchronize protein expression and treated with 2.5 mg/ml of NHS—S—S-dPEG4-biotin (Quanta Biodesign) in PBS for 30 min at 4° C. Cells were washed with DPBS and lysed with DH buffer (50 mM HEPES, 100 mM NaCl, 0.005 g/ml dodecyl maltoside), containing a protease inhibitor cocktail (Sigma), and clarified by centrifugation. Samples were then incubated overnight at 4° C. on a rotary wheel with streptavidin-agarose (Thermo Scientific). The next day, samples were centrifuged at 5000 rpm for 5 min, and the pellet (biotinylated proteins) was washed and processed for western blot analysis (using anti-HRC rabbit sera^{1,17}).

Plaque enlargement assay. Vero-SLAM cells were infected with MeV IC323-EGFP at a multiplicity of infection (MOI)=0.001. After 6 h, cells were either untreated or treated with serial dilutions of supernatant fluids from hybridomas producing either anti-F mAb 77.4 or mAb Y503. Images were acquired 5 days post-infection using immunofluorescence. Quantification was determined using fluorescent spectroscopy.

In vivo experiments: Cotton rats. Inbred cotton rats (Sigmodon hispidus) were purchased from Envigo, Inc., Indianapolis. Both male and female cotton rats aged 5 to 7 weeks were used. For infection, 10⁵ TCID50 of MeV (strain WTFb) was inoculated intranasally to isofluorane-anesthetised cotton rats in a volume of 100 μl. Treatment was performed in the same way at the indicated time points. Four days after infection, the animals were euthanized using CO2 inhalation, and their lungs were collected and weighed. Lung tissue was minced with scissors and homogenized with a glass dounce homogenizer. Vero-SLAM cells were inoculated with serial 10-fold dilutions of supernatant fluids to assess the presence of infectious virus in 48-well plates using cytopathic effect (CPE) as an endpoint. Plates were scored for CPE microscopically after 7 days, and the TCID50 was calculated as described previously⁴³.

Synergy analysis. Dose-response curves and synergy analysis were performed using the Combenefit software package, and the results were visualized using MATLAB software⁴⁴.

Statistical analysis. Statistical analysis was performed with GraphPad Prism 5 (GraphPad Software Inc. La Jolla, CA, USA) software. All data are expressed as the mean±SEM of at least three independent experiments in triplicate and analyzed using the unpaired student t-test or ANOVA, post-hoc tests when required, or the Mann-Whitney U-test. Data were considered significant when p<0.05.

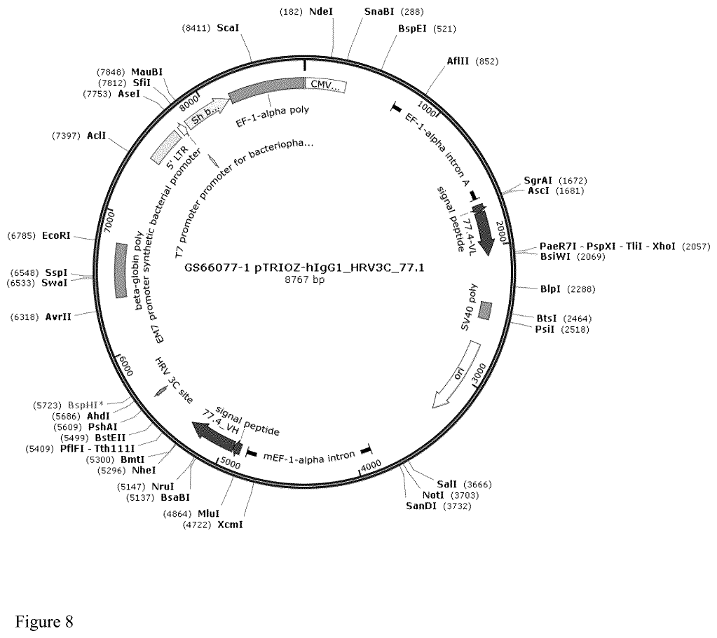

### Example 8: Grafting 77.1 mAb. See FIG. 8

The 77.1 mAb, with variable domains (VL and VH) derived from 77.4 (mouse) was cloned in pTRIOZ-hIgG1 vector. FIG. 8.

77.4 VL Sequence
(SEQ ID NO: 7)
GATGTTCAGATCACACAGAGCCCAAGCTACCTCGCAGCATCTCCAGGCG
AGACTATCACTATCAACTGTAGAGCCTCCAAGTCCATTTCTAAGTATCT
GGCTTGGTATCAGGAGAAGCCAGGTAAGACCAACGAGCTGCTGATCTAC
TCTGGAAGCACACTCCAGTCCGGTATCCCATCCAGATTCAGAGGTTCTG
GTTCTGGCACCGATTTCACCCTGACAATCAGCTCTCTGGAGCCTGAAGA
TTTCGCAATGTACTATTGTCAGCAGCATAACGAGTATACCCTGACTTTC
GGTGGAGGAACAAAGCTCGAGCTGAAGCGT
77.4 VH Sequence
(SEQ ID NO: 8)
GACGTGCAGCTCCAAGAGAGCGGTCCTGGTCTGGTGAAGCCTTCTCAAA
GCCTGTCCCTGACCTGTACCGTTTCTGGCTATAGCATCACTTCTGACTA
CGCCTGGAACTGGATTAGGCAGTTTCCTGGTAACAAGCTGGAATGGATG
GGCTACATTAGCTACACCCTGACTACAGGTTACAATCCCTCTCTGAAGT
CTAGGATTTCTATCACTCGCGACTCCTCTAAGAACCAGTTCTTCCTGCA
ACTGAATAGCGTGACAACAGAAGATACTGCTACATACTACTGTGCAAGA
TCCGGTTGGCTGCTGCCTTACTGGTACTTCGATGTGTGGGGTGCCGGCA
CAACCGTGACTGTCAGCTCC

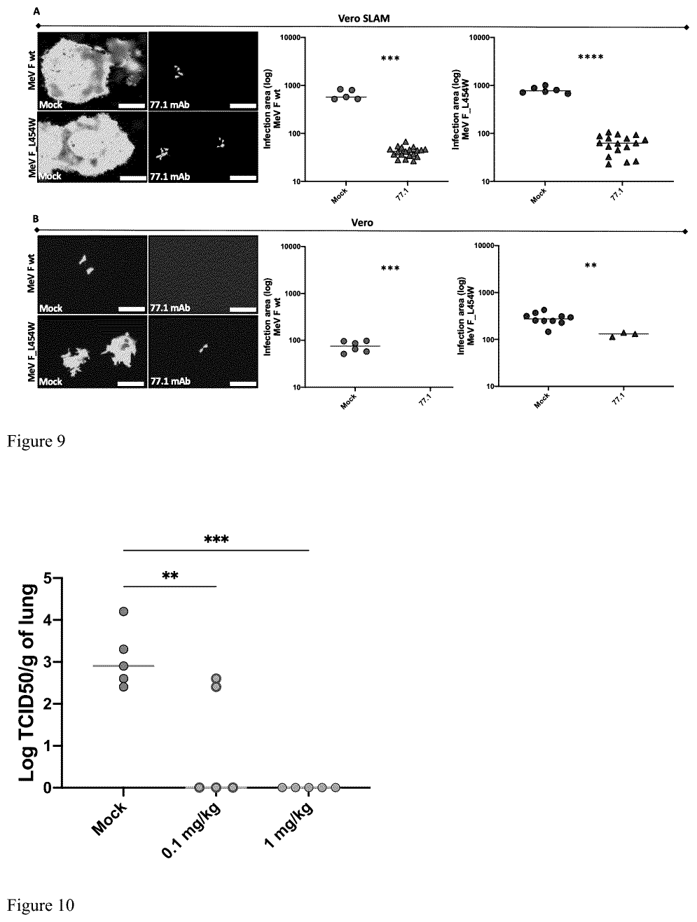

### Example 9: 77.1 mAb Inhibits Spread by MeV Wild Type (Wt) and CNS-Adapted Variant. See FIGS. 9 and 10

The antiviral activity of 77.1 mAb was assessed against MeV B3-eGFP-F wt and F_L454W (CNS-adapted variant) live virus infection (FIG. 9). Since the virus bearing L454W F spreads in the absence of a known MeV entry receptor, we compared the inhibitory efficacies in Vero cells expressing SLAM/CD150 (Vero-SLAM) (FIG. 9 Panel A) and Vero cells (FIG. 9 Panel B) that express no known MeV receptor. After a 120-min infection with either wt B3-eGFP or B3-eGFP-F L454W viruses, cells were treated with the antibody and visualized at several time points (24, 48, and 72 h). Without treatment, the two viruses spread and formed large syncytia after 72 h in Vero-SLAM cells. In Vero cells (without receptor), MeV wt did not spread, while MeV B3-eGFP-F L454W formed syncytia that were smaller than those formed in the presence of CD150/SLAM. The 77.1 mAb treatment blocked both viruses in the presence or absence of receptor.

In vivo prophylaxis of 77.1 mAb. Antiviral prophylactic efficacy for MeV in vivo was assessed in cotton rats (CR) as an infection model. The 77.1 mAb was administrated by subcutaneous delivery 12 hours before infection. Four days after infection, the animals were euthanized and the viral titers in the animals' lungs were quantified. Treatment with 77.1 mAb significantly reduced the viral titer in the lungs compared with the untreated control (See FIG. 10).

Inhibition of viral spread—see FIG. 9. Effect of human monoclonal antibody (77.1 mAb) on the areas of plaques formed by the indicated recombinant MeV viruses with eGFP in infected Vero SLAM (A) or Vero cells (B). The mAb were added after the 120-min adsorption period. Representative images of Vero SLAM after 72 h post-infection were taken using a Cytation 5 Cell Imaging Multi-Mode Reader (BioTek) (bars, 200 μm). Areas of infection were measured using BioTek Gen5 software on images randomly acquired from one experiment in duplicate. Statistical analysis was performed using the Unpaired t test with Welch's correction (**, p<0.05; ***p<0.005; ****, p<0.0001).

77.1 mAb inhibits MeV infection in vivo—see FIG. 10. Cotton rats were intranasally infected with 10⁵ TCID₅₀/ml MeV B3-eGFP wild type. Animals received either the indicated amount of the mAb 77.1 (n=5) or vehicle (n=5) 12 hours prior infection. Animals were euthanized 5 days after infection. The results are presented as TCID₅₀/g of lung tissue.

Statistical analysis was performed using the one-way ANOVA with Dunnett's multiple comparison test (**p=0.005, ***p=0.0003).

### Example 9 Material and Methods

Recombinant 77.1 mAb Expression and Purification (Creative Biolabs, CBL). The gene for the 77.1 (human monoclonal antibody) mAb derives from 77.4 mAb (mouse monoclonal antibody) and it was synthesized using PCR-based methods and cloned into the pTRIOZ-hIgG1 vector. The expression vectors of the antibody were transient transfected and expressed in mammalian cells with chemically defined culture media. The mAb was purified by affinity chromatography, ultrafiltrated and then subjected to 0.2 micron sterile filtration to get the bulk of high purity.

Viral spread inhibition in vitro. Vero or Vero-SLAM cells were plated in 12-well plates (2×10⁵ cells/well). The following day, cells were infected either with MeV B3-eGFP-F wild type and MeV B3-eGFP-F L454W (200 PFU/well for Vero-SLAM and 500 PFU/well for Vero cells) for 2 h at 37° C. The medium was replaced with complete medium and human monoclonal antibody referred as 77.1 mAb (100 ug/ml for Vero-SLAM and 10 ug for VERO cells). After 72 h pictures were obtained using a Cytation 5 Cell Imaging Multi-Mode Reader (BioTek). Areas of infection were measured using BioTek Gen5 software on images randomly acquired from one experiment in duplicate. (Unpaired t test with Welch's correction).

In vivo experiments, cotton rats. Inbred cotton rats (Sigmodon hispidus) were purchased from Envigo, Inc., Indianapolis. Both male and female cotton rats aged 5 to 7 weeks were used. For i.n. infection, 10⁵ TCID₅₀ of MeV B3-eGFP-F wild type, in PBS was inoculated intranasally to isoflurane-anesthetized cotton rats in a volume of 100 μl. Four days after infection, the animals were euthanized by CO₂ inhalation, and their lungs were collected and weighed. Lung tissue was minced with scissors and homogenized with a glass dounce homogenizer. Serial 10-fold dilutions of supernatant fluids were assessed for the presence of infectious virus in 48-well plates using cytopathic effect (CPE) in Vero SLAM cells as the endpoint. Plates were scored for CPE microscopically after 7 days. The TCID₅₀ was calculated as described previously.

### Example 10: 77.1 mAb Demonstrates Viral Neutralizing Activity In Vitro and In Vivo

We assessed the neutralization properties of mouse monoclonal antibodies that specifically target the MeV F protein in its pre-fusion state. From the most potent antibody we derived a human monoclonal antibody, referred to as 77.1 mAb, and we show that it has viral neutralizing activity in vitro and in vivo.

Grafting 77.1 mAb. The 77.1 mAb, with variable domains (VL and VH) derived from 77.4 (mouse) was cloned in pTRIOZ-hIgG1 vector. See FIG. 8.

### Results

77.1 mAb inhibits spread of MeV wild type (wt) and central nervous system (CNS)-adapted MeV variant. The antiviral activity of 77.1 mAb was assessed in MeV B3-eGFP-F wt and F_L454W (CNS-adapted variant) live virus infection (FIGS. 9A and 9B). Since the virus bearing L454W F spreads in the absence of a known MeV entry receptor, we compared the inhibitory efficacies in Vero cells expressing the known receptor SLAM/CD150 (Vero-SLAM) (FIG. 9A) and Vero cells (FIG. 9B) that express no known MeV receptor. After a 120-min infection with either MeV F_wt or F_L454W viruses, cells were treated with the antibody and visualized at several time points (24, 48, and 72 h). Without treatment, the two viruses spread and formed large syncytia after 72 h in Vero-SLAM cells. In Vero cells (without receptor), MeV F_wt did not spread, while MeV F_L454W formed syncytia that were smaller than those formed in the presence of CD150/SLAM. The 77.1 mAb treatment blocked both viruses in the presence or absence of receptor.

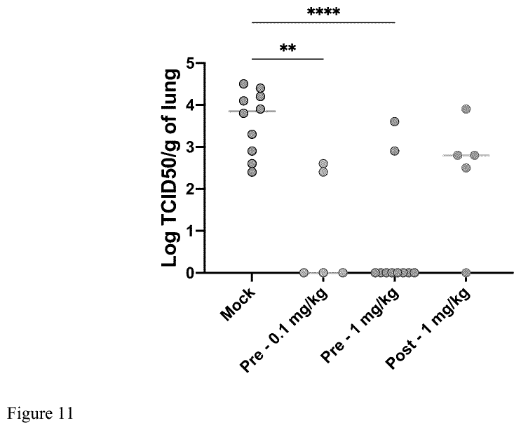

In vivo prophylaxis and post-exposure efficacy of 77.1 mAb. Antiviral efficacy for MeV in vivo was assessed in cotton rats (CR) as an infection model. The 77.1 mAb was administered subcutaneously 12 hours before infection (0.1 mg/kg or 1 mg/kg) or 24 hours post infection (1 mg/kg). Four days after infection, the animals were euthanized and the viral titers in the animals' lungs were quantified. Treatment with 77.1 mAb reduced the viral titer in the lungs compared with the untreated control (FIG. 11).

### Example 10 Materials & Methods

Recombinant 77.1 mAb Expression and Purification (Creative Biolabs, CBL). The gene for the 77.1 (human monoclonal antibody) mAb derives from 77.4 mAb (mouse monoclonal antibody) and it was synthesized using PCR-based methods and cloned into the pTRIOZ-hIgG1 vector. The expression vectors of the antibody were transiently transfected and expressed in mammalian cells with chemically defined culture media. The mAb was purified by affinity chromatography, then ultrafiltration, and then subjected to 0.2 micron sterile filtration.

Viral spread inhibition in vitro. Vero or Vero-SLAM cells were plated in 12-well plates (2×10⁵ cells/well). The following day, cells were infected either with MeV B3-eGFP-F wild type and MeV B3-eGFP-F L454W (200 PFU/well for Vero-SLAM and 500 PFU/well for Vero cells) for 2 h at 37° C. The medium was replaced with complete medium and humanized monoclonal antibody referred to as 77.1 mAb (100 ug/ml for Vero-SLAM and 10 ug for VERO cells). After 72 h pictures were obtained using a Cytation 5 Cell Imaging Multi-Mode Reader (BioTek). Areas of infection were measured using BioTek Gen5 software on images randomly acquired from one experiment in duplicate. (Unpaired t test with Welch's correction).

In vivo experiments in cotton rats. Inbred cotton rats (Sigmodon hispidus) were purchased from Envigo, Inc., Indianapolis. Both male and female cotton rats aged 5 to 7 weeks were used. For i.n. infection, 10⁵ TCID₅₀ of MeV B3-eGFP-F wild type in PBS was inoculated intranasally to isoflurane-anesthetized cotton rats in a volume of 100 μl. Four days after infection, the animals were euthanized by CO₂ inhalation, and their lungs were collected and weighed. Lung tissue was minced with scissors and homogenized with a glass dounce homogenizer. Serial 10-fold dilutions of supernatant fluids were assessed for the presence of infectious virus in 48-well plates using cytopathic effect (CPE) in Vero SLAM cells as the endpoint. Plates were scored for CPE microscopically after 7 days. The TCID₅₀ was calculated as described previously.

### Prophetic Example 11: Treatment of Other Viruses

This same technology may be used to treat or prevent other viral infections. A single-chain variable fragment (scFv) that binds to a virus F peptide, wherein the scFv blocks F-mediated membrane fusion and spread of the virus, is effective strategy against a wide range of viruses, including without limitation Nipah virus, Human parainfluenza virus, and Respiratory Syncytial Virus (RSV). A scFv that binds to the F protein of any one these viruses may be used, for non-limiting example, to prevent or treat a viral infection associated with Nipah virus, Human parainfluenza virus, or Respiratory Syncytial Virus (RSV).

### REFERENCES

1. Moss, W. J. & Griffin, D. E. Measles. Lancet 379, 153-164 (2012).
2. Simons, E., et al. Assessment of the 2010 global measles mortality reduction goal: results from a model of surveillance data. Lancet 379, 2173-2178 (2012).
3. Perry, R. T., et al. Progress toward regional measles elimination—worldwide, 2000-2014. MMWR Morb Mortal Wkly Rep 64, 1246-1251 (2015).
4. Jansen, V. A., et al. Measles outbreaks in a population with declining vaccine uptake. Science 301, 804 (2003).
5. Tatsuo, H., Ono, N., Tanaka, K. & Yanagi, Y. SLAM (CDw150) is a cellular receptor for measles virus. Nature 406, 893-897 (2000).
6. Muhlebach, M. D., et al. Adherens junction protein nectin-4 is the epithelial receptor for measles virus. Nature 480, 530-533 (2011).
7. Noyce, R. S., et al. Tumor cell marker PVRL4 (nectin 4) is an epithelial cell receptor for measles virus. PLoS Pathog 7, e1002240 (2011).
8. Chang, A. & Dutch, R. E. Paramyxovirus fusion and entry: multiple paths to a common end. Viruses 4, 613-636 (2012).
9. Harrison, S. C. Viral membrane fusion. Nature structural & molecular biology 15, 690-698 (2008).
10. Hashiguchi, T., et al. Structures of the prefusion form of measles virus fusion protein in complex with inhibitors. Proc Natl Acad Sci USA 115, 2496-2501 (2018).
11. Welsch, J. C., et al. Fatal measles virus infection prevented by brain-penetrant fusion inhibitors. Journal of virology 87, 13785-13794 (2013).
12. Sun, A., et al. Nonpeptide inhibitors of measles virus entry. J Med Chem 49, 5080-5092 (2006).
13. Doyle, J., et al. Two domains that control prefusion stability and transport competence of the measles virus fusion protein. J Virol 80, 1524-1536 (2006).
14. Plemper, R. K., et al. Design of a small-molecule entry inhibitor with activity against primary measles virus strains. Antimicrob Agents Chemother 49, 3755-3761 (2005).
15. Lambert, D. M., et al. Peptides from conserved regions of paramyxovirus fusion (F) proteins are potent inhibitors of viral fusion. Proc Natl Acad Sci USA 93, 2186-2191 (1996).
16. Figueira, T. N., et al. In Vivo Efficacy of Measles Virus Fusion Protein-Derived Peptides Is Modulated by the Properties of Self-Assembly and Membrane Residence. Journal of virology 91(2017).
17. Mathieu, C., et al. Prevention of measles virus infection by intranasal delivery of fusion inhibitor peptides. Journal of virology 89, 1143-1155 (2015).
18. Jurgens, E. M., et al. Measles fusion machinery is dysregulated in neuropathogenic variants. mBio 6(2015).
19. Malvoisin, E. & Wild, F. Contribution of measles virus fusion protein in protective immunity: anti-F monoclonal antibodies neutralize virus infectivity and protect mice against challenge. J Virol 64, 5160-5162 (1990).
20. Fayolle, J., Verrier, B., Buckland, R. & Wild, T. F. Characterization of a natural mutation in an antigenic site on the fusion protein of measles virus that is involved in neutralization. J Virol 73, 787-790 (1999).
21. Avila, M., et al. Molecular determinants defining the triggering range of prefusion F complexes of canine distemper virus. Journal of virology 88, 2951-2966 (2014).
22. Angius, F., et al. Analysis of a Subacute Sclerosing Panencephalitis Genotype B3 Virus from the 2009-2010 South African Measles Epidemic Shows That Hyperfusogenic F Proteins Contribute to Measles Virus Infection in the Brain. Journal of virology 93, e01700-01718 (2019).
23. Mathieu, C., et al. Measles Virus Bearing Measles Inclusion Body Encephalitis-Derived Fusion Protein Is Pathogenic after Infection via the Respiratory Route. Journal of virology 93, e01862-01818 (2019).
24. Ader, N., et al. Mechanism for active membrane fusion triggering by morbillivirus attachment protein. Journal of virology 87, 314-326 (2013).
25. Wangchuk, S., et al. Measles and rubella immunity in the population of Bhutan, 2017. Vaccine 37, 6463-6469 (2019).
26. Venkat, H., et al. Measles Outbreak at a Privately Operated Detention Facility: Arizona, 2016. Clin Infect Dis 68, 2018-2025 (2019).
27. Trentini, F., Poletti, P., Melegaro, A. & Merler, S. The introduction of ‘No jab, No school’ policy and the refinement of measles immunisation strategies in high-income countries. BMC Med 17, 86 (2019).
28. Toffolutti, V., McKee, M., Melegaro, A., Ricciardi, W. & Stuckler, D. Austerity, measles and mandatory vaccination: cross-regional analysis of vaccination in Italy 2000-14. Eur J Public Health 29, 123-127 (2019).
29. Patel, M. K., et al. Progress Toward Regional Measles Elimination—Worldwide, 2000-2018. MMWR Morb Mortal Wkly Rep 68, 1105-1111 (2019).
30. Patel, M., et al. Increase in Measles Cases—United States, Jan. 1-Apr. 26, 2019. MMWR Morb Mortal Wkly Rep 68, 402-404 (2019).
31. Patel, M., et al. National Update on Measles Cases and Outbreaks—United States, Jan. 1-Oct. 1, 2019. MMWR Morb Mortal Wkly Rep 68, 893-896 (2019).
32. Patel, D., Simons, H. & Schlagenhauf, P. The march of measles—Can travel medicine halt the pace? Travel Med Infect Dis 29, 4-5 (2019).
33. Nyathi, S., et al. The 2016 California policy to eliminate nonmedical vaccine exemptions and changes in vaccine coverage: An empirical policy analysis. PLoS Med 16, e1002994 (2019).
34. Mina, M. J., et al. Measles virus infection diminishes preexisting antibodies that offer protection from other pathogens. Science 366, 599-606 (2019).
35. Melegaro, A. Measles vaccination: no time to rest. Lancet Glob Health 7, e282-e283 (2019).
36. Mehtani, N.J., Rosman, L. & Moss, W. J. Immunogenicity and Safety of Measles Vaccine in HIV-infected Children: an Updated Systematic Review. Am J Epidemiol (2019).
37. Durrheim, D. N., et al. A dangerous measles future looms beyond the COVID-19 pandemic. Nat Med (2021).
38. Czuppon, P., et al. Success of prophylactic antiviral therapy for SARS-CoV-2: Predicted critical efficacies and impact of different drug-specific mechanisms of action. PLoS Comput Biol 17, e1008752 (2021).
39. Nyanguile, O. Peptide Antiviral Strategies as an Alternative to Treat Lower Respiratory Viral Infections. Front Immunol 10, 1366 (2019).
40. Moosmann, P. & Rusconi, S. Alpha complementation of LacZ in mammalian cells. Nucleic Acids Res 24, 1171-1172 (1996).
41. Porotto, M., et al. Molecular determinants of antiviral potency of paramyxovirus entry inhibitors. Journal of virology 81, 10567-10574 (2007).
42. Porotto, M., et al. Inhibition of Nipah virus infection in vivo: targeting an early stage of paramyxovirus fusion activation during viral entry. PLoS Pathog 6, e1001168 (2010).
43. Figueira, T. N., et al. In vivo efficacy of measles virus fusion protein-derived peptides is modulated by properties of self-assembly and membrane residence. Journal of virology (2016).
44. Di Veroli, G. Y., et al. Combenefit: an interactive platform for the analysis and visualization of drug combinations. Bioinformatics 32, 2866-2868 (2016).

What is claimed is:

1. A single-chain variable fragment (scFv) that binds to a measles virus (MeV) F peptide, wherein the scFv blocks F-mediated membrane fusion and spread of the MeV.

2. The scFv of claim 1, wherein the scFv interacts synergistically with a MeV fusion inhibitory peptide.

3. The scFv of claim 2, wherein the MeV fusion inhibitory peptide is HRC4.

4. A method of preventing or treating measles in a subject in need thereof, comprising administering to the subject a scFv of claim 1.

5. The method of claim 4, wherein the scFv is co-administered with a MeV fusion inhibitory peptide HRC4.

6. The method of claim 4, wherein the scFv is administered with one or more pharmaceutically acceptable excipients.

7. The method of claim 6, wherein the scFv is administered per airway.

8. The method of claim 7, wherein scFv is administered intranasally.

9. The method of claim 6, wherein the scFv is administered subcutaneously.

10. The scFv of claim 1, comprising SEQ ID NO:1.

11. The scFv of claim 2, comprising SEQ ID NO:2.

12. The scFv of claim 1 comprising SEQ ID NO:3.

13. The method of claim 5, wherein the HRC4 comprises SEQ ID NO:4.

14. The method of claim 4, for treating measles in a subject having an extant measles infection.

15. The method of claim 9, for preventing measles in a subject.

16. A single-chain variable fragment (scFv) that binds to a virus F peptide, wherein the scFv blocks F-mediated membrane fusion and spread of the virus.

17. The scFv of claim 16, wherein the virus is one or more of the group consisting of Nipah, Human parainfluenza, and Respiratory Syncytial Virus (RSV).

18. A method of preventing or treating a viral infection in a subject in need thereof, comprising administering to the subject a scFv of claim 17, wherein the viral infection is caused by Nipah, Human parainfluenza, or Respiratory Syncytial Virus (RSV).
